(19) United States
(12) Patent Application Publication
(10) Pub. No.: US 2025/0295894 A1
(43) Pub. Date: Sep. 25, 2025

(54) BALLOON CATHETER-STENT STRUCTURE COMBINATION

(71) Applicant: Alexander RUEBBEN, Monaco (MC)

(72) Inventor: Alexander RUEBBEN, Monaco (MC)

(21) Appl. No.: 18/863,344

(22) PCT Filed: May 5, 2023
(86) PCT No.: PCT/EP2023/061915

§ 371 Date: Nov. 6, 2024

(30) Foreign Application Priority Data
May 6, 2022 (DE) 10 2022 111 350.7

(51) Int. Cl. A61M 25/10; A61F 2/07; A61F 2/86; A61F 2/958; A61L 31/02; A61L 31/14

(52) U.S. Cl. CPC A61M 25/10; A61F 2/07; A61F 2/86; A61F 2/958; A61L 31/022; A61L 31/14

(57) ABSTRACT

The invention relates to a combination of a balloon catheter (1), through which a lumen extends in the longitudinal direction, with a balloon (2) being arranged in the distal section of the balloon catheter (1), said balloon being expandable by applying pressure with a fluid conducted through the lumen, and a stent structure (5), which comprises of braided wires or interconnected struts (7), wherein the stent structure (5) can be mounted on the balloon (2) of the balloon catheter (1) and can be brought into an expanded form by expansion of the balloon (2), wherein the stent structure (5) is elastic and designed such that, in the absence of external forces acting on the stent structure (5), it assumes a reduced-diameter form in which it is firmly seated on the balloon (2). With the help of the combination according to the invention, particularly persistent and hard-to-treat stenoses can be effectively dilated.

The invention relates to a combination of a balloon catheter, through which a lumen extends in the longitudinal direction, with a balloon being arranged in the distal section of the balloon catheter, which can be expanded by exerting pressure caused by a fluid introduced through said lumen, and a stent structure which is made up of braided wires or interconnected struts, said stent structure being designed for mounting on the balloon of the balloon catheter and brought into an expanded form as a result of the expansion of the balloon.

Using balloon catheters is nowadays standard practice in everyday clinical routine. Their use in the context of intravascular interventions involves as a rule the widening of constricted vessel areas, either by means of the balloon catheter itself or in combination with other medical devices such as balloon-expandable stents, for example. In the event of percutaneous transluminal angioplasty (PTA) or percutaneous transluminal coronary angioplasty (PTCA) for the coronary vessel region a balloon catheter is navigated to the site of the stenosis using a guidewire and a guiding catheter and expanded by the introduction of a fluid under pressure (approx. 4 to 12 bar). Deposits present in the area of the stenosis are pressed into the vessel wall. Moreover, a stent (vascular endoprosthesis) may be placed in position with a view to keeping the blood vessel permanently open. To prevent a stenosis from recurring due to vasoconstrictive overgrowth of the widened site, drug-eluting balloon catheters can also be put to use, which are designed to dispense a drug such as paclitaxel at the site of vasoconstriction during expansion. When the treatment has been completed and the balloon has subsequently been folded up, the balloon catheter is withdrawn from the vascular system and removed. In many cases, balloon angioplasty has replaced conventional bypass surgery.

Frequently chosen as access site for balloon catheters is the femoral artery in the groin area. As it extends, at least partially, relatively near the surface, the femoral artery is thus easily accessible for the attending physician, both for treatments to be performed in the coronary area, also, however, for other areas of the vascular system such as the brain or extremities. In addition, catheters of relatively large lumen with large outer diameters can be inserted through the femoral artery (inguinal artery).

Atherosclerosis is a commonly occurring lifestyle disease and the cause of a large number of deaths worldwide. It is associated with atherosclerotic plaques, which can be composed of calcium salts and have a relatively hard consistency, as well as fibrotic tissue, forming on the inner walls of arteries. As a result, the blood vessels narrow to such an extent that blood flow is severely restricted. State-of-the-art technology includes so-called cutting balloons or scoring balloons for widening such stenoses. These balloons are designed for use with balloon catheters and have metal wires attached to their outer surface. As the balloon expands, these metal wires penetrate into the atherosclerotic plaques and cause them to break open. This treatment approach is of particular significance in the area of the coronary arteries or peripheral areas such as the leg arteries.

A balloon catheter of this type which is known as the Barath cutting balloon, is described in publication U.S. Pat. No. 5,196,024. The cutting edges provided on the balloon are conducive to causing longitudinal incisions in the vessel walls in the process of balloon expansion, enlarging the vessel diameter and preventing cell proliferation or restenosis, which is a common occurrence with conventional angioplasty. Such longitudinal cutting is performed in fibrotic or calcified tissue. Due to the cutting edges being secured to the balloon catheter both distally and proximally, they follow the expansion and deflation movement of the balloon.

US 2005/0245864 A1 discloses a cutting balloon by means of which incisions are made in fibrotic or calcified tissue not radially evenly, but in a certain radial area of the inner vessel wall, for which purpose the balloon is attached eccentrically to the axis of the guidewire. In this way, it is avoided that unaffected areas of the inner wall of the blood vessel become damaged.

Scoring and cutting balloons have acquired a place in arterial dilation, but in the majority of cases, conventional balloon catheters are still used. A disadvantage of using scoring or cutting balloons, however, is that they represent a significant modification of a conventional balloon catheter and, in case of doubt, have to be kept available by the clinic in addition to conventional balloon catheters.

It is thus the objective of the present invention to provide the attending physician with a flexible option for modifying a balloon catheter in such a way that its effectiveness at widening problematic stenoses improves.

As proposed by the invention this objective is achieved by a combination comprising
a balloon catheter, through which a lumen extends in the longitudinal direction, with a balloon being arranged in the distal section of the balloon catheter, which can be expanded by the application of pressure using a fluid conducted through the lumen, and
a stent structure which is composed of braided wires or interconnected struts, wherein the stent structure can be mounted on the balloon of the balloon catheter and, by expansion of the balloon, can be brought into an expanded shape, wherein the stent structure is elastic and is designed such that, without external forces acting on the stent structure, it assumes a reduced-diameter shape in which it is firmly seated on the balloon.

Other than the scoring or cutting balloons already described hereinbefore, the invention makes use of a typical balloon catheter. On the balloon portion a stent structure is mounted, usually as a separate object, which is composed of braided wires or interconnected struts. In the process of balloon expansion, the stent structure also widens resulting in the struts/wires being pressed into the inner wall of the vessel. Especially in the presence of heavily fibrotic or calcified tissue, this enables the vascular stenosis to be opened up, and appropriately supports balloon dilation.

In contrast to the conventional placement of a stent with the help of a balloon catheter, the stent structure used as proposed by the invention is not intended to permanently remain in the blood vessel, but rather is intended to only aid in the widening of the blood vessel and shall subsequently be removed. For this reason, the stent structure is preset in such a way that it “wants” to take on a contracted or compressed shape in which it sits firmly on the balloon in such a way that a proximal retraction of the balloon together with the stent structure can take place. The stent structure can thus be brought into an expanded form by application of external forces, that is, by the expansion of the balloon, in which the structure exerts the described effect on the inner wall of the vessel. However, as soon as the balloon contracts again, the stent structure also assumes a contracted form having a significantly smaller outer diameter, in which it rests on the deflated balloon. This allows the balloon to be then retracted in the proximal direction together with the stent structure resting on the deflated balloon and removed from the vascular system.

From prior art, a plurality of stents is known that have a secondary structure imprinted on them, which the stent adopts when no external forces are acting on it. These are self-expanding stents, which as a rule consist of a metal having shape memory properties. Nevertheless, the basic characteristic of the stent in these cases is exactly opposite to what the invention proposes. Self-expanding stents are designed to widen or unfold on their own as soon as they are released from a catheter that has previously prevented them from expanding and kept them in a compressed state. They will then remain in the blood vessel to keep it open permanently. In contrast to this, the stent structure provided in accordance with the invention is intended to contract or reduce its diameter by itself and is only caused to expand when external forces are applied, that is, when the expansion of the balloon takes place. As soon as these forces no longer act, the stent structure contracts to re-assume its diameter-reduced form. Unlike self-expanding stents, the structure is not supposed to remain in the blood vessel permanently, but to be removed together with the balloon catheter.

In order for the stent structure to assume its intended contracted, diameter-reduced shape by itself, it must exhibit sufficient elasticity to allow it to contract again after expansion. It is particularly beneficial if the stent structure has superelasticity, which is also known as pseudoelasticity. Pseudoelastic alloys, also known as shape memory alloys, are characterized by their property of returning to their original shape when the load is removed even if they have been considerably deformed. In comparison, the expansion of pseudoelastic alloys is ten times that of conventional spring steels without the alloys suffering permanent deformation. Accordingly, materials having shape memory properties can be conducively employed for the stent structure. Therefore, particularly advantageous is a stent structure which is at least partially made of a shape memory material, especially a shape memory alloy.

In particular, nickel-titanium alloys have proven their worth as shape memory alloys, for example Nitinol, or ternary nickel-titanium-chromium alloys or nickel-titanium-copper alloys. However, other shape memory materials, for example other alloys or even shape memory polymers, are also conceivable.

The use of cobalt-chromium or cobalt-chromium-nickel alloys is also possible. Alloys of this type are also frequently employed in the field of medical engineering. In particular, it is possible to use an alloy known as Elgiloy, which consists of 39 to 41% w/w cobalt, 19 to 21% w/w chromium, 14 to 16% w/w nickel, 11.3 to 20.5% w/w iron, 6 to 8% w/w molybdenum, 1.5 to 2.5% w/w manganese and max. 0.15% w/w carbon. Furthermore, stainless steel may also be employed.

Nevertheless, the stent structure does not have to be composed of a single material; it is also possible to combine different materials, especially different metals and alloys. It is thus conceivable to manufacture individual wires or struts of the stent structure from one material and make use of a different material for other wires or struts. For example, individual wires/struts may be made of radiopaque materials which enables the attending physician to visualize the treatment. Platinum and platinum alloys such as platinum-iridium or tantalum can be chosen as radiopaque materials.

It is also an option to employ wires/struts that consist of several materials. For example, so-called DFT® (drawn filled tubing) wires, are known in prior art in which the core of the wire is made of a material that differs from the core surrounding sheath. A wire or strut may, for example, comprise a core made of an X-ray visible material and a sheath made of a material having shape memory properties. Radiopaque or X-ray visible materials may in particular be those mentioned hereinbefore. DFT® wires are available from the company of Fort Wayne, for example.

With respect to the stent structure forming part of the inventive combination and being referred to herein as stent structure, it is to be noted, however, that this term is actually meant to define the buildup of the object and not its function. Conventional stents are implants provided to remain in the blood vessel permanently and keep it open. On the other hand, in the case of the stent structure proposed by the present invention, the stent structure is to be removed from the vascular system after the treatment and widening of the blood vessel has been completed. Nevertheless, with a view to preventing restenosis a conventional stent can be placed in the area of the dilated stenosis after balloon dilatation has been performed.

In the case of stents and similarly constructed medical objects, a distinction is usually made between interwoven and cut structures. Interwoven structures are composed of individual wires that form a wire braiding. This means, the braiding can easily assume both an expanded shape as well as a diameter-reduced shape. The expansion or widening movement is usually associated with the stent structure being significantly shortened, while contraction is associated with the structure being significantly elongated.

Another category of stents includes structures that comprise interconnected struts. These stents are usually produced by laser cutting, i.e., a laser is used to cut the desired structure out of a tube. The interconnected struts form a lattice structure comprising a plurality of cells delimited by the struts. The cells can be closed, that is, they are completely surrounded by struts, and/or can be open. In particular, individual cells can be closed and other cells left open.

As regards the stent structure proposed by the invention, both variants can be employed, that is, interwoven structures made of individual wires as well as (laser) cut structures comprising individual struts. Other options for manufacturing a stent structure are also conceivable, for example 3D printing.

The wires or struts of the stent structure are preferably shaped in such a way that they can effectively penetrate into the deposits and proliferations present on the inner walls of the vessels. For this purpose, the wires/struts can have a round or oval cross-section, but it is also possible to provide a cross-section for the wires/struts that tapers radially outwards. For example, the wires/struts may have a triangular cross-section, with one edge of the wires/struts facing radially outwards to enable blade-like penetration into the deposits/proliferations. A triangular cross-section shall also be understood to define a cross-section in which the lateral surfaces of the wire/strut are rounded to a greater or lesser extent. Also conceivable are wires or struts having a rectangular, square or trapezoidal cross-section, with the lateral surfaces also having a rounded configuration in this case as well.

Within the context of the invention, proximal is understood to mean in the direction of the exterior of the body, i.e., toward the attending physician, while distal shall be understood to denote the opposite direction, that is, toward the target site. The longitudinal direction is the direction from proximal to distal or vice versa. Radial refers to the plane perpendicular to the longitudinal axis of the balloon catheter.

According to one embodiment of the invention, the stent structure and the balloon catheter are designed so as to form separate components. This enables the treating physician to decide individually, depending on the nature of the stenosis, whether dilation of the vessel with the balloon catheter alone is sufficient or whether the stent structure provided by the invention should additionally be arranged on the balloon of the balloon catheter. In the event of a relatively easy-to-widen stenosis, the physician will usually refrain from using the additional stent structure; however, in case of doubt or if a particularly persisting stenosis is encountered, for example one that is heavily fibrotic or calcified, he or she will opt to place or crimp the stent structure onto the balloon of the balloon catheter. Since the stent structure has a small cross-section in its inactive state, which it takes on when no external forces act on it, the structure is positioned closely against the deflated balloon catheter. This means that it is possible both to simultaneously advance the stent structure on the balloon in the distal direction when inserting the balloon catheter and to simultaneously retract the stent structure in the proximal direction when withdrawing the balloon catheter.

According to an alternative embodiment, however, one end of the stent structure could also be secured to the balloon catheter, while the remaining portion of the stent structure extends over the balloon. Typically, the structure is secured at a position immediately adjacent to the balloon, i.e., immediately proximal or distal to the balloon. In this case, the proximal end of the stent structure is preferably secured to the balloon catheter, that is, proximally attached to the balloon of the balloon catheter. In this embodiment, the unattached end of the stent structure is freely movable and therefore can be freely expanded and contracted. In this context, the term “end of the stent structure” shall be understood to refer not only to the outermost end of the stent structure in the longitudinal direction; it is sufficient if the stent structure is attached to the balloon catheter in the area of one end, which does not preclude small parts of the stent structure from extending further proximally or distally beyond the point of attachment.

In this embodiment, the stent structure is affixed to the balloon catheter, thereby preventing the stent structure from becoming detached or slipping relative to the balloon catheter when the balloon catheter is either moved forward in distal direction or retracted in proximal direction. On the other hand, the physician is less flexible in this embodiment with regard to their decision as to whether the treatment should be carried out with or without the stent structure in place.

Expediently, the outer diameter of the stent structure is about two to four times larger in the expanded form than in the reduced-diameter form. Accordingly, this ensures, on the one hand, that the stent structure in its reduced-diameter state has a small cross-section, so that the balloon catheter with the stent structure placed on it can easily be moved also through narrow blood vessels, while, on the other hand, however, the cross-section of the stent structure in expanded state makes sure the stenosis can be effectively eliminated. A typical diameter of the stent structure in the reduced-diameter form ranges between 0.8 and 0.9 mm for the coronary area and between 0.8 and 1.5 mm for the peripheral area, and a typical diameter of the stent structure in the expanded form ranges between 2 and 3 mm for the coronary area and between 2 and 6 mm for the peripheral area. Generally speaking, the exact dimensions significantly depend on the type of blood vessel in which the balloon catheter is to be deployed. Accordingly, the lumen of peripheral arteries in the lower leg, for example, is larger than that of the arteries located in the cardiovascular or neurovascular area.

The balloon catheter typically comprises a shaft having a lumen for the supply of the fluid passing through it. As a rule, a second lumen extends at least partially through the shaft in the longitudinal direction, with said lumen serving to accommodate a guidewire. This lumen may extend up to the distal end of the balloon catheter, that is, the guidewire may exit the balloon catheter at its distal end. Accordingly, a guidewire can first be moved to the desired target position before the balloon catheter itself is pushed forward to the target position via the guidewire.

In this context, essentially two different systems are known from prior art, namely over-the-wire (OTW) and rapid exchange (Rx) balloon catheters. The balloon catheter proposed by the invention can be either an OTW or an Rx balloon catheter. Whereas in an OTW catheter the lumen for the guidewire extends over the entire length of the catheter from proximal to distal, an Rx catheter is provided with a separate guidewire access port (Rx port) where the guidewire exits the catheter significantly distal to the proximal end of the catheter. Accordingly, in the case of an OTW balloon catheter, the lumens for the fluid supply and the guidewire run concentrically or parallel to each other from the proximal end of the catheter up to the balloon, whereas in the case of an Rx catheter, this is only the case between the Rx port and the balloon. The section between the Rx port and the proximal end, on the other hand, has only one lumen for the fluid supply. The lumens may be concentric in the areas where the catheter has two lumens, so that the narrower inner lumen accommodating the guidewire passes through the wider outer lumen via which fluid delivery takes place.

The term balloon as it is used within the scope of the present invention shall be understood to define the element of a balloon catheter that can be expanded by feeding in a fluid, irrespective of the shape or material of said expandable element. Basically, balloon catheters are sufficiently known from prior art and comprise an elongated shaft extending from proximal to distal as well as a balloon which is arranged in the distal section. As regards its dimensions such a catheter is suitably designed for the insertion into the respective blood vessel system. The exact dimensions of such catheters may vary depending on whether the blood vessel, for example, is a coronary artery, an intracranial blood vessel or an artery in the lower leg. Moreover, the balloon catheter is provided with means for delivering a fluid to the balloon, said means comprising a delivery/supply lumen extending over the length of the balloon catheter. The lumens extending through the shaft are usually each formed by a hose-like or tubular tube.

In this context, the term tube is used to denote a conduit or hose that extends at least partially through the balloon catheter in longitudinal direction and has a lumen running through the interior of the tube. The tube can have the shape of a hollow cylinder of circular or elliptical cross-section, but this is not a mandatory requirement. As far as the cross-section is concerned, almost any other shape is also conceivable. However, a circular or perhaps elliptical cross-section is to be viewed as an advantage since one tube can be easily passed through the other tube, usually running the second tube through the first tube.

In the event of a compliant or semi-compliant balloon, the balloon may be made at least in part of an elastic material. As elastic material, a polyurethane, a polyolefin copolymer, a polyethylene or a silicone may, for example, be used. Other materials that can be employed are thermoplastic elastomers, in particular polyether block amides (PEBA). This is a thermoplastic elastomer obtainable by poly-condensation of a carboxylic acid polyamide with a polyether having terminal OH groups. In particular, PEBA is available on the market under the tradename of PEBAX® by the Arkema company. Polyamides such as nylon (polyhexamethylene adipamide) or those offered for sale under the tradename of Grilamid® by the company of EMS-GRIVORY may also be employed.

The pressure applied to the balloon to bring about its expansion in the blood vessel typically ranges between 4 and 12 bar, preferably between 6 and 8 bar. The fluid to be used may, for example, be water mixed with contrast medium or a saline solution mixed with contrast medium. The dimensions of the balloon may vary greatly depending on the field of application; for example, the diameter in the expanded state may range from approx. 1 mm to approx. 50 mm, and the length may range between approx. 5 mm and approx. 300 mm. However, the dimensions may also deviate from this, for example, when using the balloon/balloon catheter for applications in urology or veterinary medicine.

The shaft of the balloon catheter may be made of customary materials, although different materials may also be put to use, for example, to make the distal section softer than the proximal section. Commonly used materials are polymers such as polyethylene, polyurethane, polyvinyl chloride, polyamides, polyimides, silicones, polyetheramides, polytetrafluorethylene, EPDM (ethylene-propylene-diene rubber), polyether block amides (PEBA) or polyamides marketed under the tradename of Grilamid® by the company EMS-GRIVORY. If though expedient, in particular the proximal areas of the shaft can also be made of metal, e.g., stainless steel.

The length of the shaft, including the area where the balloon is located but excluding hubs or similar connectors proximal to the shaft, is typically at least 90 cm. Accordingly, it is possible to insert the balloon catheter via the femoral artery in the groin region and advance it to different locations in the vascular system.

If so desired, the balloon catheter may be provided with a balloon that is coated with an active substance (drug eluting balloon). The active agent is preferably selected from the group consisting of tretinoin, orphan receptor agonists, elafin derivatives, corticosteroids, steroid hormones, paclitaxel, rapamycin (sirolimus), tacrolimus, hydrophobic proteins, heparin and/or hormone-like or cell proliferation-modifying substances. Particularly preferred are paclitaxel, tacrolimus and sirolimus. Mixtures of these active substances may also be used. Moreover, derivatives of the above cited active agents may also be of use, wherein said derivatives may in particular be salts, esters, and amides. As steroid hormones methylprednisolone, dexamethasone or estradiol may be used, for example.

At the proximal end of the balloon catheter, adjoining the shaft, a so-called catheter hub is usually arranged, i.e., a connector that serves to attach the fluid supply and pressurization device. The connection can be a conventional Luer or Luer Lock connector, for example. It is particularly expedient to provide two Luer lock connectors, typically female connectors, one for connecting the first lumen to a balloon dilator and another for inserting the guidewire into the balloon catheter. The connectors may, for example, be made from a polycarbonate. At its proximal end the guidewire extending through the balloon catheter may be held by means of a torquer which facilitates handling the usually very thin guidewire.

Over the length of the balloon catheter and/or stent structure radiopaque markers may be arranged at various positions, said markers serving the purpose of making the catheter visible on radiographs. In particular, said markers may be manufactured of platinum or a platinum alloy.

As described, the inventive balloon catheters are preferably used in blood vessels, particularly in the field of angioplasty. In this case, the target site of the balloon is a blood vessel, with relevant blood vessels may be located in different areas, in particular in cardiovascular, neurovascular and peripheral locations, wherein the combination proposed by the invention being particularly suitable for cardiovascular and peripheral applications. However, the inventive combination of balloon catheter and stent structure may also be utilized in other medical fields. A field of application includes urology, where balloon catheters are inserted into the urinary bladder as bladder catheters. The catheter is fixed via the balloon. In this case, the balloon may, for instance, be coated with a substance such as heparin to prevent bacterial colonization and incrustation.

In pulmonology, balloon catheters can be applied to dilatate or occlude a bronchus. Balloon catheters can also be employed in the field of gynecology. In the field of orthopedics, balloon catheters can be put to use for the treatment of vertebral fractures with a view to realigning the vertebrae by means of balloon expansion techniques (balloon kyphoplasty). In principle, the balloon catheter/stent structure combination proposed by the invention can be put to use in all fields of medicine where balloon catheters are employed, with the balloon catheter being of particular importance for insertion into blood vessels and for the treatment of atherosclerosis.

Further elucidation of the invention is provided through the enclosed figures by way of examples, where

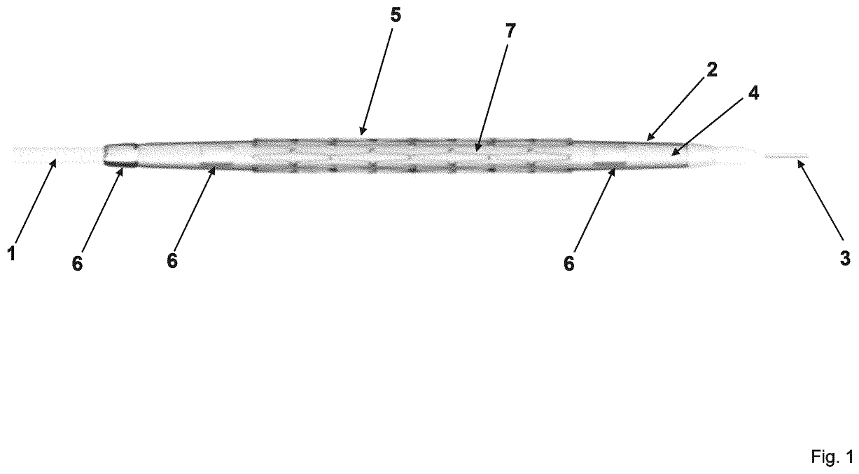

FIG. 1 shows a stent structure arranged on the balloon catheter in the folded state;

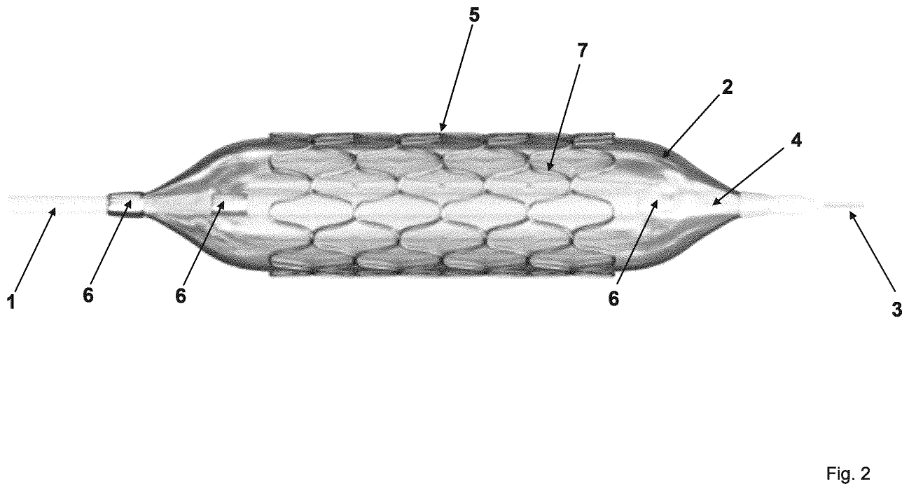

FIG. 2: displays a stent structure arranged on the balloon catheter in the expanded state; and

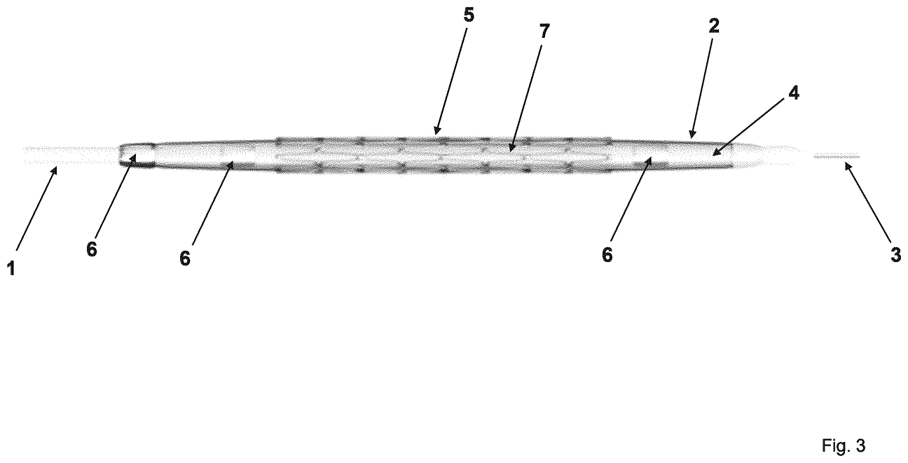

FIG. 3 shows a stent structure on the balloon catheter after deflation of the balloon.

FIG. 1 is an illustration as a side view of the combination of balloon catheter 1 and stent structure 5 according to the invention, with proximal being on the left and distal on the right in the representation chosen here. The distal section of balloon catheter 1 shown here has a balloon 2 arranged in it, whereas the largest part of the proximally located shaft of balloon catheter 1 is not shown. The balloon catheter 1 exhibits a lumen 4 that extends in the longitudinal direction for the accommodation of a guidewire 3 reaching in the distal direction beyond the balloon catheter 1. Concentrically arranged to this lumen 4 is a further lumen, not shown here, through which a fluid can be introduced into the balloon 2. Moreover, the balloon catheter 1 features radiopaque markers 6 that make it possible for the treating physician with the aid of imaging techniques to visualize the correct placement of the balloon catheter 1 in the area of a stenosis.

Mounted in place or crimped onto balloon 2 is a stent structure 5, which is illustrated in FIG. 1 in its diameter-reduced form. This means that it can be navigated to the target position together with the balloon catheter 1. Stent structure 5 in this case is produced by laser cutting, said structure being composed of interconnected struts 7 that form a lattice structure being comprised of a plurality of cells.

FIG. 2 illustrates the same combination of balloon catheter 1 and stent structure 5 but with the balloon 2 in expanded state causing stent structure 5 to expand appropriately and thus be able to exert forces on the inner wall of the vessel. This result in the struts 7 of the stent structure 5 to cut and penetrate into calcifications and fibrotic tissue in the area of the stenosis and break them open.

Illustration as per FIG. 3 finally illustrates the combination comprising balloon catheter 1 and stent structure 5 after balloon 2 has been deflated, i.e., the fluid expanding balloon 2 has been withdrawn. The elastic design characteristics of the stent structure 5 bring about a recontraction of the stent structure 5 resulting in the structure to again assume a diameter-reduced shape. This causes the stent structure 5 to once again be firmly seated on the balloon 2 so that it can be retracted in proximal direction together with balloon catheter 1.

1. Combination comprising
a balloon catheter, through which a lumen extends in the longitudinal direction, with a balloon being arranged in the distal section of the balloon catheter, which can be expanded by the application of pressure using a fluid conducted through the lumen, and
a stent structure which is made up of wires that are braided or of struts that are interconnected, it being possible for the stent structure to be applied to the balloon of the balloon catheter and, by the balloon expanding, to be brought into an expanded form, wherein
the stent structure is elastic and is designed such that, without external forces acting on the stent structure, it assumes a diameter-reduced shape in which it is firmly seated on the balloon.

2. Combination according to claim 1, wherein the stent structure is at least partially composed of a shape memory material.

3. Combination according to claim 2, wherein the stent structure is at least partially composed of a nickel-titanium or nickel-titanium-copper alloy.

4. Combination according to claim 1, wherein the stent structure is at least partially composed of a cobalt-chromium or cobalt-chromium-nickel alloy.

5. Combination according to claim 1, wherein the wires or struts of the stent structure have a round or oval cross-section.

6. Combination according to claim 1, wherein the wires or struts of the stent structure have an edge pointing radially outwards.

7. Combination according to claim 1, wherein the wires or struts of the stent structure have a triangular cross-section.

8. Combination according to claim 1, wherein one end of the stent structure is fixed to the balloon catheter.

9. Combination according to claim 8, wherein the proximal end of the stent structure is fixed to the balloon catheter.

10. Combination according to claim 1, wherein the outer diameter of the stent structure in the expanded form is two to four times as large as in the diameter-reduced form.

11. Combination according to claim 1, wherein the stent structure in the diameter-reduced form has an outer diameter ranging between 0.8 and 1.5 mm.

12. Combination according to claim 11, wherein the stent structure in the diameter-reduced form has an outer diameter of 0.8 to 0.9 mm.

13. Combination according to claim 1, wherein the stent structure in the expanded form has an outer diameter ranging between 2 and 6 mm.

14. Combination according to claim 13, wherein the stent structure in the expanded form has an outer diameter of 2 to 3 mm.
